(19) United States
(12) Patent Application Publication
(10) Pub. No.: US 2013/0323309 A1
(43) Pub. Date: Dec. 5, 2013

(54) Sustained Release Composition of Memantine

(71) Applicants: Pravin Meghrajji Bhutada, Pune (IN); Ashish Ashokrao Deshmukh, Pune (IN); Sajeev Chandran, Pune (IN); Vineeth Raghavan, Pune (IN); Shirishkumar Kulkarni, Pune (IN)

(72) Inventors: Pravin Meghrajji Bhutada, Pune (IN); Ashish Ashokrao Deshmukh, Pune (IN); Sajeev Chandran, Pune (IN); Vineeth Raghavan, Pune (IN); Shirishkumar Kulkarni, Pune (IN)

(73) Assignee: LUPIN LIMITED, Mumbai (IN)

(21) Appl. No.: 14/000,264

(22) PCT Filed: Feb. 7, 2012
(86) PCT No.: PCT/IB12/50539

§ 371 Date: Aug. 19, 2013

(30) Foreign Application Priority Data
Feb. 17, 2011 (IN) 201/KOL/2011

(51) Int. Cl. A61K 9/50; A61K 31/13

(52) U.S. Cl. CPC A61K 9/5073; A61K 31/13
USPC 424/490; 514/662

(57) ABSTRACT

A sustained release pharmaceutical composition comprising; a core including memantine or its pharmaceutically acceptable salts and one or more pharmaceutical acceptable excipients, and a sustained release coating comprising a water insoluble substance and a water soluble substance where the ratio of the water insoluble substance to the water soluble substance is from about 1:0 to about 3:5:1, optionally containing an immediate release coating having memantine where the immediate release coating is applied over the sustained release coating.

### FIELD OF THE INVENTION

This invention relates to sustained release pharmaceutical composition of memantine.

### BACKGROUND OF THE INVENTION

Memantine (1-amino-3,5-dimethyladamantane) is an analogue of 1-amino-cyclohexane which is a systemically-active uncompetitive NMDA receptor antagonist having moderate affinity for the receptor, strong voltage dependency and rapid blocking/unblocking kinetics.

Memantine and other 1-aminoalkylcyclohexanes have proven useful in alleviation of various progressive neurodegenerative disorders such as dementia in patients with moderate to severe AD, parkinson's disease, and spasticity.

Memantine immediate release tablet is marketed in US by Forest under trade name Namenda® and in Europe by Merz under trade name Axura® and Ebixa®

Memantine immediate release marketed formulations are mostly given twice daily, however switching to once daily sustained release formulation offers improved patient compliance and control of symptoms due to less frequent dosing and may provide an improved side-effect profile.

Memantine, or a pharmaceutically acceptable salt thereof, is highly soluble in aqueous media and polar solvents. The high solubility of memantine makes it difficult to achieve a consistent drug release rate over extended periods. A further problem with highly water soluble drugs formulated into a sustained release dosage form is that a significant and variable “burst” of drug can occur from these systems.

Sustained release formulations for drugs have become increasingly available. This is true especially when the particular drug is relatively soluble. It is typically the goal of all sustained-release preparations to provide a longer period of pharmacological response after the administration of the dosage form than that which is ordinarily experienced after the administration of the immediate release dosage forms. Desirably the sustained release provides a generally uniform and constant rate of release over an extended period of time, which achieves a stable and desired blood (plasma) level of the active ingredient without the need for frequent administration of the medicament.

The commercially available (approved by US FDA) extended release composition of memantine is Namenda XR capsules (Forest Laboratories, Inc. and Merz Pharmaceuticals GmbH).

In the prior art, many techniques have been used to provide sustained and extended-release pharmaceutical compositions of memantine in order to maintain therapeutic levels of medicaments and to minimize the effects of missed doses of drugs caused by a lack of patient compliance.

Some of sustained release compositions for memantine described in prior art are as follows:

U.S. Pat. No. 6,194,000 (Smith et al) discloses a method for the therapeutic treatment of pain related to wind up in a human or animal. The method of the invention is practiced by administering to the subject an effective amount of an analgesic pharmaceutical composition which includes a NMDA receptor antagonist in an immediate release form combined with an NMDA receptor antagonist in a sustained release form.

U.S. Pat. No. 5,382,601 (Nurnberg et al) discloses solid pharmaceutical dosage forms containing memantine which exhibit an extended two-phase release profile, the matrix of this formulation contains both a water-soluble and a water-insoluble salt of casein, preferably sodium and calcium caseinate.

US 201000266684 (went et al) discloses solid pharmaceutical composition comprising an extended release form of memantine in pellet containing a capsule dosage form wherein the composition comprises coated cores having about 8%-16.5% by weight a sustained release coating comprising ethyl cellulose and a porosity enhancer selected from the group consisting of PVP and HPMC a plasticizer, and an ethyl cellulose to porosity enhancer ratio of about 8:2 to 9:1.

However, there is still an existing and continual need for a once a day sustained release composition containing memantine, or a pharmaceutically acceptable salt thereof with release rate over a targeted period of time. Accordingly, the present invention provides a sustained release composition of memantine or its pharmaceutically acceptable salts thereof.

### OBJECTS OF THE INVENTION

The object of the present invention is a sustained release pharmaceutical composition comprising; a core comprising memantine or its pharmaceutically acceptable salts and one or more pharmaceutical acceptable excipient(s), and a sustained release coating comprising the water insoluble substance and a water soluble substance wherein ratio of the water insoluble substance to the water soluble substance is from about 1:0 to about 3.5:1, optionally containing an immediate release coating comprising memantine over the sustained release coating.

Yet another object of the invention is a sustained release pharmaceutical composition of memantine wherein about more than 15% drug is released in 1 hour, about 0-50% of drug released in about 4 hours, about 60-90% of drug released in about 8 hours, and more than about 80% of drug released in about 10 to12 hours following entry into a use environment.

### DETAILED DESCRIPTION OF THE INVENTION

One embodiment relates to,

a sustained release pharmaceutical composition comprising:
a. a core comprising memantine or its pharmaceutically acceptable salts and one or more pharmaceutical acceptable excipient(s),
b. a sustained release coating comprising a water insoluble substance and a water soluble substance wherein ratio of the water insoluble substance to the water soluble substance is from about 1:0 to about 3.5:1.

“Sustained release” according to present invention means drug delivery system releasing the drug at a predetermined rate, locally or systemically, for a specified period of time. Sustained release can be used interchangeably with prolonged release, programmed release, timed release, extended release, controlled release, and modified release, slow release and other such dosage forms.

The “pharmaceutical composition” according to present invention may be but not limited to powders, pellets, beads, granules, tablets, compacts, capsules, microcapsules, tablets in capsules, tablets in tablets, microspheres, shear form particles, floss, and flakes or mixtures thereof. Tablets include but not limited to single layered tablets, multilayered tablets, mini tablets, bioadhesive tablets, caplets, matrix tablets, tablet within a tablet, mucoadhesive tablets.

The term “memantine” according to present invention is meant to cover memantine in the form of freebase or its pharmaceutically acceptable salt, hydrate(s), solvate(s) and physiologically functional derivative(s) and precursors thereof. The term also includes all polymorphic forms, whether crystalline or amorphous in nature.

The amount of memantine present, inclusive of pharmaceutically acceptable salts thereof, preferably from about 5 mg to about 30 mg.

The “core” according to the invention can be constituted according to different principles.

a) inert substance layered with memantine, optionally mixed with other pharmaceutically acceptable excipients, can be used as the core material for the further processing or

b) memantine can be mixed with other pharmaceutically acceptable excipients to construe the core which can be immediate release and/or sustained release core.

The inert substance can be but not limited to different oxides, celluloses, organic polymers, different inorganic salts, sugars, non-pareils, alone or in mixtures. Before the seeds are layered, the active substance may be mixed with one or more other pharmaceutically acceptable excipients. The core can be homogenous or have an internal structure comprising pellets, tablets, minitablets, capsules, granules, beads or pills. The term “inert” is a pharmaceutically substance which is inactive in relation to the active ingredient and other pharmaceutically acceptable excipient(s), that is to say, it does not react with the active ingredient and other pharmaceutically acceptable excipient(s) in the conditions used in such a way that there is decomposition thereof.

Before the inert substance is layered, memantine may be mixed with one or more pharmaceutically acceptable excipient(s). Such excipient(s) may be but not limited to binder, surfactant, filler, disintegrating agent, solvent alone or in mixtures.

The immediate release core comprises memantine admixed with one or more pharmaceutically acceptable excipient(s) such as but are not limited to binders, fillers or diluents, lubricants, glidants and surface-active agents.

The sustained release core comprises memantine admixed with but not limited to waxes (eg. carnauba, bees wax, paraffin wax, ceresine, shellac wax, fatty acids, fatty alcohols), oils, hardened oils or fats (eg. hardened rapeseed oil, castor oil, beef tallow, palm dil, soya bean oil), polymers (eg. hydroxypropyl cellulose, polyvinylpyrrolidone, polyvinyl acetate, hydroxypropylmethyl cellulose(hypromellose), ethylcellulose, polyethylene glycol) and other pharmaceutically acceptable excipient(s) known to those familiar with the art.

The core according to present invention may be powders, pellets, beads, granules, tablets, compacts, capsules, microcapsules, tablets in capsules, tablets in tablets, microspheres, shear form particles, floss, and flakes or mixtures thereof. Tablets include but not limited to single layered tablets, multilayered tablets, mini tablets, bioadhesive tablets, caplets, matrix tablets, tablet within a tablet, mucoadhesive tablets.

The core may be formulated following any conventional techniques known in the art, namely dry granulation, aqueous or non-aqueous wet granulation, melt granulation, direct compression, roller compaction, pelletization or layering, melting, intimate and non-intimate blending, kneading, extrusion and the like.

The term “comprising” which is synonymous with “including”, “containing”, or “characterized by” here is defined as being inclusive or open-ended, and does not exclude additional, unrecited elements or method steps, unless the context clearly requires otherwise.

As used herein, “pharmaceutically acceptable salts” refer to derivatives of the memantine wherein memantine is modified by reacting it with an acid or base as needed to form an ionically bound pair. Examples of pharmaceutically acceptable salts include conventional non-toxic salts or the quaternary ammonium salt of the parent compound formed, for example, from non-toxic inorganic or organic acids. Suitable non-toxic salts include those derived from inorganic acids such as hydrochloric, hydrobromic, sulfuric, sulfamic, phosphoric, nitric, and others known to those of ordinary skill in the art. The salts prepared from organic acids such as amino acids, acetic, propionic, succinic, glycolic, stearic, lactic, malic, tartaric, citric, ascorbic, pamoic, maleic, hydroxymaleic, phenylacetic, benzoic, salicylic, sulfanilic, fumaric, oxalic, isethionic, and others known to those of ordinarily skilled in the art. The most preferred salt of memantine for the present invention is hydrochloride salt.

Pharmaceutically acceptable excipient(s) include but are not limited to binders, fillers or diluents, lubricants, glidants, surface-active agents or solvent(s) and mixtures thereof.

One excipient can perform more than one function.

All excipients can be used at levels well known to the persons skilled in the art.

Binders may be selected from, but are not limited to, starches such as potato starch, wheat starch, corn starch; microcrystalline cellulose such as products known under the registered trademarks Avicel, Filtrak, Heweten or Pharmacel; celluloses such as hydroxypropyl cellulose, hydroxyethyl cellulose, hydroxypropylmethyl cellulose, ethyl cellulose, sodium carboxy methyl cellulose; natural gums like acacia, alginic acid, guar gum; liquid glucose, dextrin, povidone, syrup, polyethylene oxide, polyvinyl pyrrolidone, poly-N-vinyl amide, polyethylene glycol, gelatin, poly propylene glycol, tragacanth, combinations thereof and other materials known to one of ordinary skill in the art and mixtures thereof.

Fillers or diluents, may be selected from, but are not limited to, carbohydrates, confectioner's sugar, compressible sugar, dextrates, dextrin, dextrose, fructose, lactitol, mannitol, sucrose, starch, lactose, xylitol, sorbitol, microcrystalline cellulose, calcium carbonate, calcium phosphate dibasic or tribasic, calcium sulphate, and the like and other materials known to one of ordinary skill in the art and combinations thereof.

Lubricants may be selected from, but are not limited to, those conventionally known in the art such as Mg, Al or Ca or Zn stearate, glyceryl behenate, mineral oil, sodium stearyl fumarate, stearic acid, talc, and the like and other materials known to one of ordinary skill in the art and combinations thereof.

Glidants may be selected from, but are not limited to, silicon dioxide, magnesium trisilicate, powdered cellulose, starch, talc, tribasic calcium phosphate, calcium silicate, magnesium silicate, colloidal silicon dioxide, silicon hydrogel and other materials known to one of ordinary skill in the art and combinations thereof.

Surface active agents may be selected from but are not limited to dioctyl sodium sulfosuccinate (DSS), triethanolamine, polyoxyethylene, sorbitan and poloxalkol derivatives, quaternary ammonium salts or other materials known to one of ordinary skill in the art and combinations thereof.

The pharmaceutical composition of the invention may also comprise disintegrant which may be selected from but are not limited to alginic acid, carboxymethylcellulose calcium, carboxymethylcellulose sodium, croscarmellose sodium, crospovidone, guar gum, magnesium aluminium silicate, sodium alginate, sodium starch glycolate and starches and other materials known to one of ordinary skill in the art and combinations thereof.

Solvents may be used in present invention include all the solvents well known in the art or their mixtures thereof.

The core of memantine is further coated with sustained release coating comprising a water insoluble substance and water soluble substance wherein ratio of the water insoluble substance to the water soluble substance is from about 1:0 to about 3.5:1.

Water-soluble substance includes but not limited to any water soluble substance or polymer which includes but not limited to alkylcelluloses such as methylcellulose; hydroxyalkylcelluloses, for example, hydroxymethyl cellulose, hydroxypropylmethyl cellulose, hydroxyethyl cellulose, hydroxypropyl cellulose and hydroxybutylcellulose; carboxyalkylcelluloses such as carboxymethylcellulose; alkali metal salts of carboxyalkylcelluloses such as sodium carboxymethylcellulose; carboxyalkyl alkylcelluloses such, carboxyalkylcellulose esters; other natural, semi-synthetic, or synthetic polysaccharides such as alginic acid, alkali metal and ammonium salts thereof, carrageenans, galactomannans, tragacanth, agar-agar, gum arabic, guar gum, xanthan gum, starches, pectins, such as sodium carboxymethyl amylopectin, chitin derivatives such as chitosan, polyfructans, inulin, sugars, lactose, sucrose, fructose, mannitol, polyvinylalcohol, polyvinylpyrrolidone, polyalkylene oxides such as polyethylene oxide and polypropylene oxide and copolymers of ethylene oxide and propylene oxide, polyethylene glycol, sodium lauryl sulphate and other materials known to one of ordinary skill in the art.

Water insoluble substance includes but not limited to any water insoluble substance or polymer which includes but not limited to cellulose derivatives as ethylcellulose, cellulose acetate, polymethacrylates containing quaternary ammonium group, high molecular weight polyvinyl alcohols, polyvinyl acetate dispersion (eg: Kollidon), waxes, hydrogenated vegetable oil, Gelucire(polyethylene glycol glycerides composed of mono-, di- and triglycerides and mono- and diesters of polyethylene glycol), purified grades of beeswax; fatty acids; long chain fatty alcohols, such as cetyl alcohol, myristyl alcohol, and stearyl alcohol; glycerides such as glyceryl esters of fatty acids like glyceryl monostearate, glyceryl distearate, glyceryl esters of hydrogenated castor oil and the like; oils such as mineral oil and the like, or acetylated glycerides; stearic acid, paraffin, carnauba wax, talc; and the stearate salts such as calcium, magnesium, zinc and, cellulose acetate butyrate or mixtures thereof and other materials known to one of ordinary skill in the art.

The sustained release coating may further comprises one or more additive(s) but not limited to such as plasticizers, fillers, polishing agents, opacifying agents, colouring agents, antitacking agents and the like.

Plasticizers may be selected from but are not limited to dibutyl sebacate, triethyl citrate, triacetin, acetylated triacetin, tributyl citrate, glycerol tributyrate, natural, semi-synthetic and synthetic glycerides, monoglyceride, acetylated monoglycerides, fractionated coconut oil, rape oil, olive oil, sesame oil, castor oil, hydrogenated castor oil, acetyltributylcitrate, acetyltriethylcitrate, glycerine, sorbitol, diethyl oxalate, diethyl phthalate, dibutyl phthalate, diethyl malate, diethyl fumarate, dibutyl succinate, diethyl malonate, dioctyl phthalate, and the like.

Fillers may be selected from but are not limited to such as talc, precipitated calcium carbonate and the like.

Polishing agents may be selected from but are not limited beeswax, carnauba wax, synthetic chlorinated wax and the like.

Opacifying agents may be selected from but are not limited to titanium dioxide, ferric oxide, sunset yellow and the like.

Colouring agents may be selected from but not limited to dyes, lakes and the like.

Antitacking agents may be selected from not limited to adipic acid, magnesium stearate, calcium stearate, zinc stearate, glyceryl monostearate, talc, silica, sodium benzoate and the like.

In another embodiment of the invention there is a seal coat between the core and the sustained release coating.

The term “seal coat” is synonymous to various terms like separating layer, seal coating layer, intermediate layer, barrier coating layer, film coating and the like. Seal coat comprises the substances but not limited to water-soluble substance, water-insoluble substance and one or more pharmaceutically acceptable excipient(s).

Another object of the invention is a sustained release pharmaceutical composition of memantine which further comprises an immediate release coating of memantine over the sustained release coating.

A suitable immediate release coating of memantine may simply comprise admixing memantine with one or more pharmaceutical excipient(s). These mixtures may be prepared by blending, mixing, dissolution and evaporation etc.

In another embodiment of the invention there is a seal coat between the immediate release and the sustained release coating.

Pharmaceutical composition of the invention can be coated by a wide variety of methods. Suitable methods include compression coating, coating in a fluidized bed or a pan and hot melt (extrusion) coating or any well known in the art.

In yet another embodiment, pharmaceutical composition of the invention can be prepared by combining immediate release granules of memantine with sustained release granules of memantine and compressed into tablets or filled in capsules.

The composition includes memantine as immediate release part in association with a sustained release part. The composition may include an amount of memantine in the immediate release part of approximately 5% to 60% of the total memantine. An immediate release content of about 10% to 30% is particularly preferred. The sustained release part of memantine may constitute the remainder of the active ingredients.

The sustained release composition of memantine may have a single phase or multiphase release profile.

The pharmaceutical composition of the invention can be formed by various methods known in the art such as by dry granulation, wet granulation (aqueous, non-aqueous, hydroalcoholic), melt granulation, direct compression, dry granulation, double compression, extrusion spheronization, layering and the like.

In the another embodiment, the process of making the pharmaceutical formulation of the invention comprises preparing a
a) a core by pelleting or layering of memantine with one or more pharmaceutically acceptable excipients.
b) coating the core with a sustained release coating consisting a mixture of water soluble and water insoluble substance wherein ratio of water insoluble substance to water soluble substance is from about 1:0 to 3.5:1.
c) optionally applying an immediate release coating of memantine over the sustained release coating.

In another embodiment, the process of making the pharmaceutical formulation of the invention comprises preparing a
a) a matrix core of memantine by mixing memantine with one or more pharmaceutically acceptable excipients.
b) coating the core with a sustained release coating consisting a mixture of water soluble and water insoluble substance wherein ratio of water insoluble substance to water soluble substance is from about 1:0 to 3.5:1.
c) optionally applying an immediate release coating of memantine over the sustained release coating.

The sustained release composition of the invention have a release rate wherein about 0-50% of drug released in about 4 hours, about 60-90% of drug released in about 8 hours and greater than about 80% of drug released in about 10-12 hours following entry into used environment

The term “entry into a used environment” means contact of a formulation of the invention with the gastro-intestinal fluids of the patient to whom it is administered, or with a fluid intended to simulate gastro-intestinal fluids (Eg: simulated gastric fluid (SGF), simulated intestinal fluid (SIF)).

The composition of the invention is suitable for the treatment of mild to moderate and moderate to severe alzheimer's disease, memory disorder, retention disorder, cognitive subjective, cognitive impairment, memory, concentration or attention problems.

The following examples are illustrative of the present invention, and the examples should not be considered as limiting the scope of this invention in any way, as these examples and other equivalents thereof will become apparent to those versed in the art, in the light of the present disclosure, and the accompanying claims.

### EXAMPLES

| | | | |
|---|---|---|---|
| | Example 1 | Example 2 | Example 3 |
| Ingredients | % w/w | % w/w | % w/w |

Pellets hardening

| | | | |
|---|---|---|---|
| Sugar sphere | 41.21 | 45.72 | 43.92 |
| Hypromellose | 1.21 | 1.35 | 1.29 |
| Polyethylene glycol | 0.12 | 0.13 | 0.13 |
| Purified Water | Qs | Qs | |

Drug loading

| | | | |
|---|---|---|---|
| Hardened sugar spheres | 42.55 | 47.21 | 45.34 |
| Memantine HCl | 14.57 | 16.16 | 15.52 |
| Talc | 1.46 | 1.62 | 1.55 |
| Hypromellose | 15.56 | 17.26 | 16.58 |
| Purified Water | Qs | qs | qs |

Seal coating

| | | | |
|---|---|---|---|
| Drug loaded pellets | 74.13 | 82.24 | 79 |
| Hypromellose | 1.52 | 1.68 | 1.62 |
| Polyethylene glycol | 0.12 | 0.13 | 0.13 |
| Purified Water | Qs | qs | qs |

Sustained release coating

| | | | |
|---|---|---|---|
| Drug loaded seal coated | 75.75 | 84.04 | 80.72 |
| pellets | | | |
| Ethyl cellulose | 9.75 | 4.24 | 7.44 |
| Sodium lauryl sulphate | — | — | 0.32 |
| Hypromellose | 3.25 | — | — |
| Triethyl citrate | 0.97 | 0.40 | 0.65 |
| Isopropyl alcohol | Qs | Qs | qs |
| Dichloromethane/Methanol | Qs | qs | qs |

Seal coating

| | | | |
|---|---|---|---|
| Extended release coated | 89.72 | 88.69 | 89.13 |
| pellets | | | |
| Hypromellose | 2.63 | 2.69 | 2.59 |
| Polyethylene glycol | 0.18 | 0.27 | 0.26 |
| Purified Water | Qs | qs | qs |

Immediate Release drug loading

| | | | |
|---|---|---|---|
| Extended release seal | 92.53 | 91.65 | 91.98 |
| coated pellets | | | |
| Memantine HCl | 2.43 | 2.69 | 2.59 |
| Hypromellose | 2.43 | 2.69 | 2.59 |
| Purified water | qs | qs | qs |

Film coating

| | | | |
|---|---|---|---|
| IR drug loaded extended | 97.39 | 97.04 | 97.15 |
| release pellets | | | |
| Hypromellose | 2.43 | 2.69 | 2.59 |
| Polyethylene glycol | 0.18 | 0.27 | 0.26 |
| Purified Water | qs | qs | qs |

Brief Manufacturing Procedure:

Pellets Hardening

1. Dissolve hypromellose and polyethylene glycol in purified water.

2. Coat the sugar sphere with above solution of step 1 for hardening.

Drug Loading

3. Dissolve memantine HCl and hypromellose in purified water.

4. Add talc to the above solution of step 3 and stir continuously.

5. Coat the above dispersion of step 4 on hardened sugar sphere of step 2 using fluidized bed drier.

Seal Coat and Extended Release Coating

6. Seal coat the drug loaded pellets of step 5 with hypromellose and polyethylene glycol solution in water.

7. Dissolve ethyl cellulose, hypromellose and triethyl citrate to the mixture of isopropyl alcohol and dichloromethane.

8. Coat the drug loaded seal coated pellets of step 6 with solution of step 7.

Seal Coat and Immediate Release Drug Loading

9. Seal coat the extended release pellets of step 8 with hypromellose and polyethylene glycol solution in water.

10. Dissolve memantine HCl and hypromellose in purified water.

11. Coat the above drug solution on seal coated pellets of step 9.

12. Films coat the step 11 with hypromellose and polyethylene glycol solution in purified water.

In-Vitro Dissolution Study

The in vitro test used to measure release rate of Memantine from a composition of the invention is as follows:

Dissolution medium—pH 1.2 (0.1 N HCl with NaCl); Volume—900 ml at 37° C.±5

Apparatus—Basket (Type-I); RPM—100 rpm

TABLE 1
In-vitro dissolution of Example 1

| Time (hr) | Cumulative % Drug Release |
|---|---|
| 0 | 0 |
| 0.5 | 10-20 |
| 1 | 10-30 |
| 2 | 10-30 |
| 4 | 25-50 |
| 6 | 50-85 |
| 8 | 60-90 |
| 10 | More than 70 |
| 12 | More than 80 |

1. A sustained release pharmaceutical composition comprising:
a. a core comprising memantine or its pharmaceutically acceptable salts and one or more pharmaceutical acceptable excipients, and
b. a sustained release coating comprising a water insoluble substance and a water soluble substance, wherein a ratio of the water insoluble substance to the water soluble substance is from about 1:0 to about 3.5:1.

2. The sustained release pharmaceutical composition of claim 1 wherein the water soluble substance is selected from hydroxylpropyl methyl cellulose, hydroxypropyl cellulose, polyvinylpyrrolidone, polyvinyl alcohol, sodium lauryl sulphate, polyethylene glycol, sugars, lactose, sucrose, fructose, mannitol, carrageenans, galactomannans, tragacanth, agar-agar, gum arabic, guar gum, xanthan gum, starches, pectins or mixtures thereof.

3. The sustained release pharmaceutical composition of claim 1 wherein water insoluble substance is selected from ethyl cellulose, cellulose acetates, polymethacrylates containing quaternary ammonium group, hydrogenated vegetable oil, gelucire, polyvinyl acetate, cellulose acetate butyrate or mixtures thereof.

4. The sustained release pharmaceutical composition of claim 1 further comprising a seal coating between the core and the sustained release coating.

5. The sustained release pharmaceutical composition of claim 1 further comprising an immediate release coating comprising memantine applied over the sustained release coating.

6. The sustained release pharmaceutical composition of claim 1 wherein the composition is used for the treatment of alzheimer's disease, memory disorder, retention disorder, cognitive subjective, cognitive impairment, memory, concentration or attention problems.

7. A process for preparation of pharmaceutical composition of memantine comprising:
a) preparing a core by pelleting or layering of memantine with one or more pharmaceutically acceptable excipients, and
b) coating the core with a sustained release coating comprising a mixture of a water soluble substance and a water insoluble substance wherein a ratio of water insoluble substance to water soluble substance is from about 1:0 to about 3.5:1.

8. A process of making a pharmaceutical formulation comprising:
a) preparing a matrix core of memantine by mixing memantine with one or more pharmaceutically acceptable excipients, and
b) coating the core with a sustained release coating consisting of a mixture of a water soluble substance and a water insoluble substance wherein ratio of the water insoluble substance to the water soluble substance is from about 1:0 to about 3.5:1.

9. A sustained release pharmaceutical composition comprising:
a) a core comprising memantine or its pharmaceutically acceptable salts and one or more pharmaceutical acceptable excipients, and
b) a sustained release coating comprising a water insoluble substance and a water soluble substance wherein a ratio of the water insoluble substance to the water soluble substance is from about 1:0 to 3.5:1,wherein about more than 15% drug is released in 1 hour, about 0-50% of drug is released in about 4 hours, about 60-90% of drug is released in about 8 hours, and more than about 80% of drug is released in about 10-12 hours following entry into used environment.

10. The process of claim 7 further comprising applying an immediate release coating of memantine over the sustained release coating.

11. The process of claim 8, further comprising applying an immediate release coating of memantine over the sustained release coating.
